(19) United States
(12) Patent Application Publication
(10) Pub. No.: US 2014/0142456 A1
(43) Pub. Date: May 22, 2014

(54) ENVIRONMENTAL AND PATIENT MONITOR FOR PROVIDING ACTIVITY RECOMMENDATIONS

(71) Applicant: Control A Plus, LLC, Arlington, VA (US)

(72) Inventors: Matthew Eric Fischer, Orem, UT (US); Anthony DiMarco, Stowe, PA (US); Daniel Nicholas Kirk, Arlington, VA (US); Rajakumaran Kuppusamy, Elmhurst, NY (US); Jacqueline Nicole Rogers, New York, NY (US); Jesse Scott Swift, Tucson, AZ (US)

(21) Appl. No.: 13/873,077

(22) Filed: Apr. 29, 2013

Related U.S. Application Data
(60) Provisional application No. 61/639,387, filed on Apr. 27, 2012.

(51) Int. Cl. A61B 5/00; A61B 5/091

(52) U.S. Cl. CPC A61B 5/7275; A61B 5/091; A61B 5/0022
USPC 600/538

(57) ABSTRACT

A patient manager provides activity recommendations to a patient based on monitored data. The patient manager monitors personal, environmental, and external data to identify baseline and personal best thresholds to determine appropriate activity recommendations for a patient that is trying to manage a disease such as asthma. A personal monitor measures a peak flow rate by having the patient blow through the personal monitor. An environmental monitor measures ambient conditions in a room and communicates the conditions with the patient manager. The patient manager also pulls in third-party regional weather information, and generates activity recommendations based on the personal, environmental, and regional information.

### CROSS-REFERENCES TO RELATED APPLICATIONS

This application claims the benefit of U.S. Provisional Patent Application No. 61/639,387 entitled “PATIENT MONITOR” and filed on Apr. 27, 2012 for Matthew Fischer, et al., which is incorporated herein by reference.

### TECHNICAL FIELD

The embodiments of the disclosure relate generally to computing devices and environmental sensors, and more specifically, relate to a method and apparatus for monitoring a user's reaction to environmental variables.

### BACKGROUND

Asthma is an inflammatory disorder of a person's airways. In those affected, the inflammation causes episodes of difficult breathing, chest tightness, and coughing. The inflammation also makes the person sensitive to allergens, irritants, smoke, etc. Every day, there are 30,000 asthma attacks in the United States alone. Of those, 9 people will pass away. Of the rest, 5,000 will go to the emergency room, and 1,000 of those will stay there for days. Children with asthma look like normal kids, but in reality they lead a very limited life. They are told not to laugh, they can't run, they can't play like the rest of the kids. This condition leaves it mark physically, emotionally and mentally on kids. Over time these kids feel powerless and beholden to their condition. They become scared to do just about anything that involves exercise or running. Their Asthma triggers become their enemies.

Preventing the onset of an asthma attack is a major goal of anyone who has asthma. Environmental factors, such as the above mentioned irritants and allergens, can be some of the strongest indicators of a susceptibility to an asthma attack. Identification of these factors (i.e., “triggers”), is critical for the successful management of asthma attacks. Adults with asthma learn to recognize when they are having a “good day” or a “bad day,” and they modify their activity accordingly. However, a child is typically incapable of successful asthma management.

### BRIEF DESCRIPTION OF THE DRAWINGS

Embodiments of the present disclosure are illustrated by way of example, and not by way of limitation, and can be more fully understood with reference to the following detailed description when considered in connection with the figures in which:

FIG. 1 illustrates one embodiment of a system for monitoring a patient;

FIG. 2 is a perspective view diagram illustrating one embodiment of the personal monitor, according to embodiments of the present disclosure;

FIG. 3 is a perspective view diagram illustrating another embodiment of the personal monitor, in accordance with one embodiment of the present disclosure;

FIG. 4 is a block diagram illustrating one embodiment of the environment monitor, in accordance with one embodiment of the present disclosure;

FIG. 5 is a schematic block diagram illustrating one embodiment of the patient manager, in accordance with embodiments of the present disclosure;

FIG. 6 is a schematic block diagram illustrating one embodiment of the user interface, in accordance with embodiments of the present disclosure;

FIG. 7 is a schematic block diagram illustrating another embodiment of the user interface in accordance with embodiments of the present disclosure;

FIG. 8 is a schematic block diagram illustrating another embodiment of the user interface, in accordance with embodiments of the present disclosure;

FIG. 9 is a schematic block diagram illustrating another embodiment of the user interface, in accordance with embodiments of the present disclosure;

FIG. 10 illustrates a flow diagram of one embodiment of a method for generating activity recommendations; and

FIG. 11 is a diagram of one embodiment of a computer system for generating patient activity recommendations.

### DETAILED DESCRIPTION

Described herein are methods and systems for an environmental and patient monitor that provides activity recommendations to the patient based on monitored data. The system monitors personal, environmental, and external data to identify baseline and personal best thresholds to determine appropriate activity recommendations for a patient that is trying to manage a disease such as asthma. The system utilizes a personal monitor that is capable of measuring a peak flow rate by having the patient blow through the personal monitor. The personal monitor communicates the peak flow rate with a patient manager. An environmental monitor measures ambient conditions in a room and communicates the conditions with the patient manager. The patient manager also pulls in third-party regional weather information, and generates activity recommendations based on the personal, environmental, and regional information.

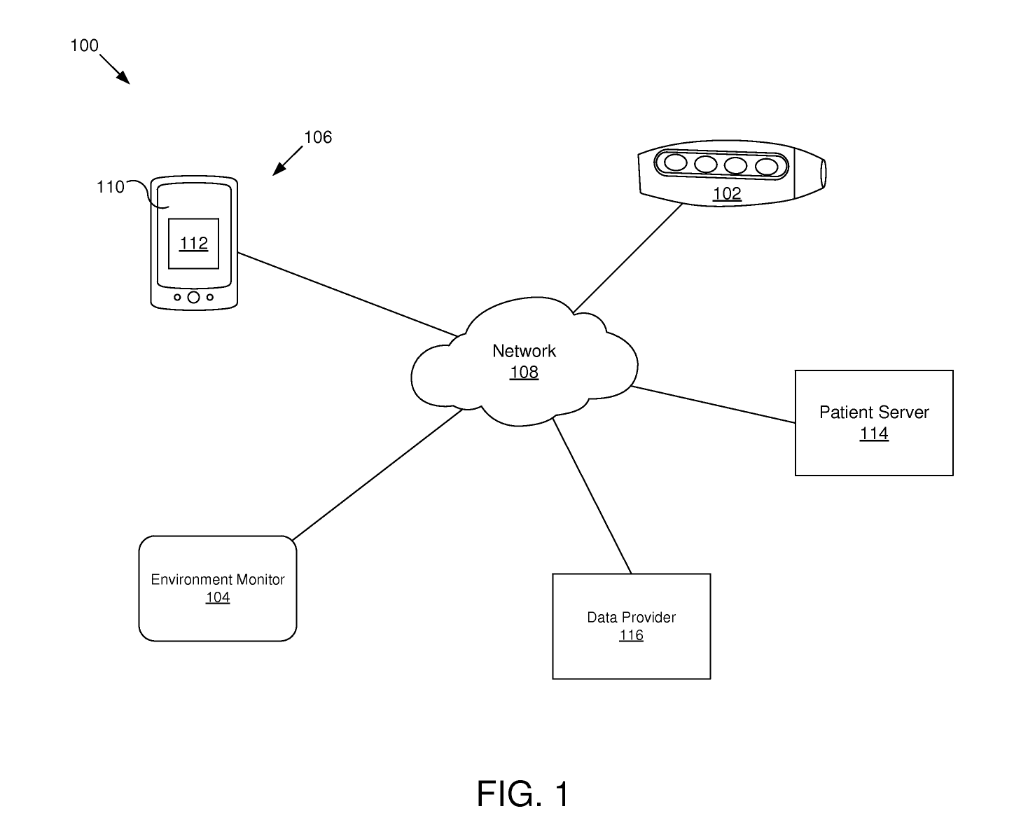

FIG. 1 illustrates one embodiment of a system 100 for monitoring a patient. The system 100, in one embodiment, is configured to monitor events and environment variables that can trigger an exacerbation or flare up of a disease. Although the below disclosure will be described with reference to asthma, the devices and methods may be applied to other diseases, such as pulmonary fibrosis, cystic fibrosis, chronic obstructive pulmonary disease, and other non-lung related diseases such as diabetes. Irritants such as air-borne particulates are causes of asthma attacks. The irritants may be plants or animal allergens, or man-made particulates (i.e., pollution). The system 100 provides education, awareness, and control of environmental variables that might lead to an asthma attack.

In one embodiment, the system 100 includes a personal monitor 102, an environment monitor 104, and a notification device 106. The personal monitor 102, the environment monitor 104, and the notification device 106 are configured to communicate over a network 108. The network 108, in one example, is a global communications network that implements a communication protocol for enabling communication between the personal monitor 102, the environment monitor 104, and the notification device 106. One example of a global communications network is the Internet. In another embodiment, the network 108 is any one of, or a combination of, WANs, LANs, WLANs, etc. The devices of the system 100 may communicate using any wired or wireless communication standard, including, but not limited to, Bluetooth, Wi-Fi, LTE, GSM/EDGE, CDMA, WiMAX, NFC, etc. In other words, the devices 102, 104, 106 may be configured to communicate over any available network 108. In another embodiment, the devices 102, 104, 106 of the system 100 are configured to establish and maintain peer-to-peer connections, or in other words, direct connections between devices without the use of the network 108.

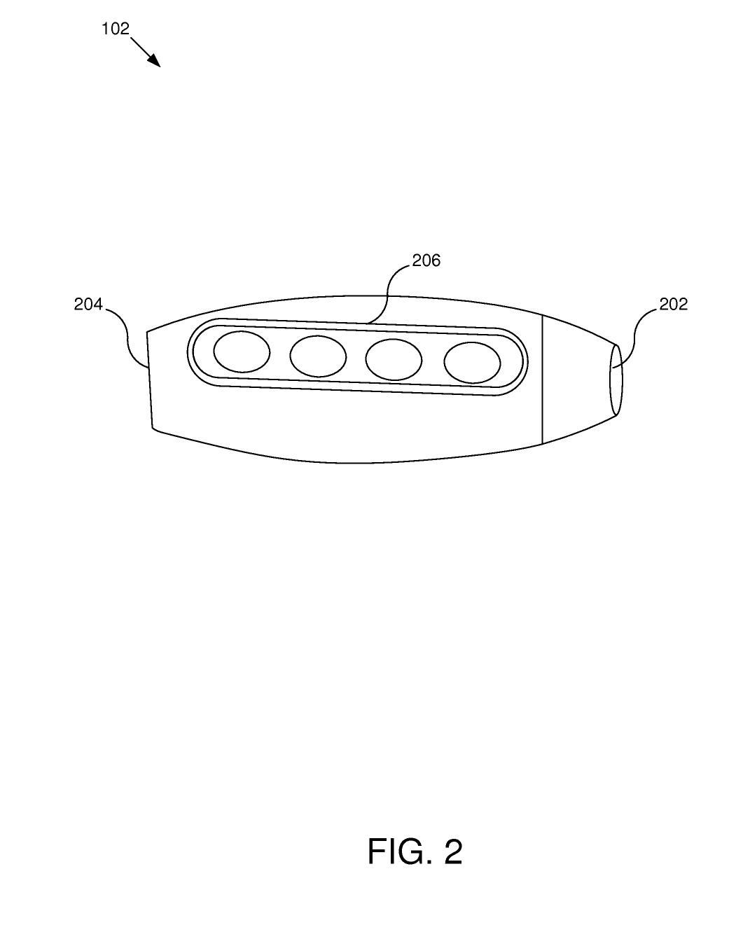

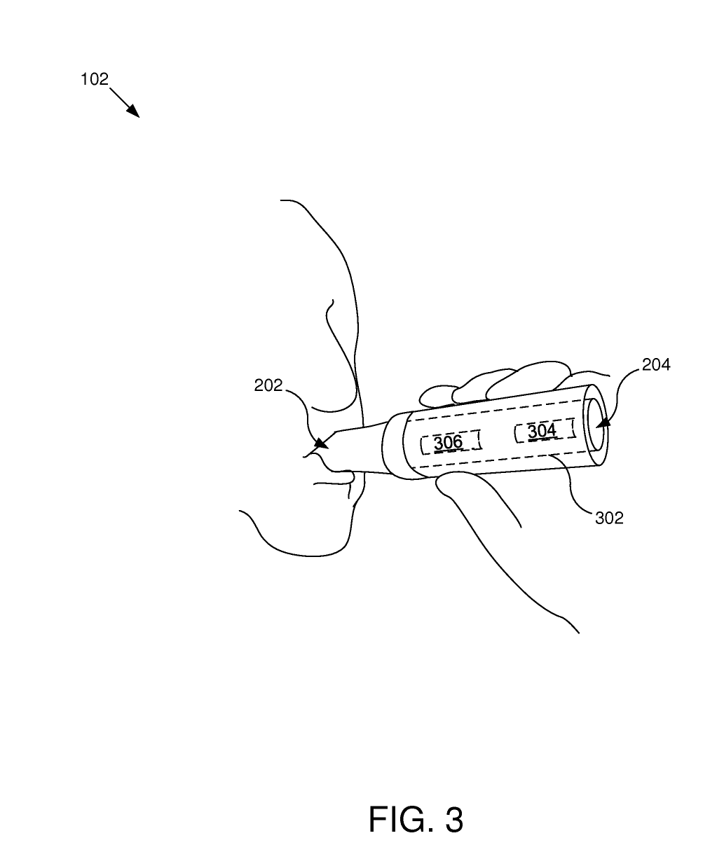

The personal monitor 102, in one embodiment, is configured to monitor personal indicators or signs that a person may be at risk of having an attack or inflammation, such as an asthma attack. In the example of asthma, the personal monitor 102 is configured with sensors for determining the lung capacity of the patient, as will be described below in greater detail. In the example of a non-lung related disease, such as diabetes, the personal monitor 102 may be configured with a blood glucose monitor. In one embodiment, the personal monitor 102 includes a peak flow meter, a network interface controller, and a data storage device. The personal monitor 102 will be discussed in greater detail below with reference to FIGS. 2 and 3

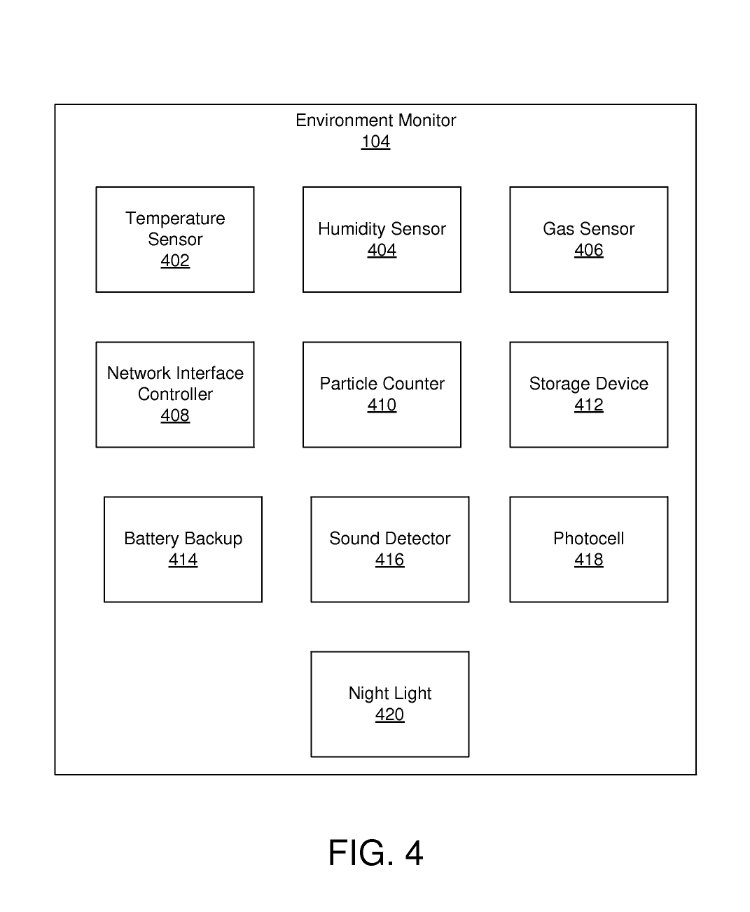

The environment monitor 104 includes sensors for monitoring environmental factors that may trigger an attack. Factors that can lead to an asthma attack can include, but are not limited to, dust, animal dander, mold, perfume, etc. Accordingly, the environment monitor 104 may be configured with a particulate sensor. The particulate sensor, in one embodiment, is a particle counter such as a laser particle counter. The laser particle counter may rely on light scattering, light obscuration, or direct imaging to determine a particulate count and size. In a further embodiment, the environment monitor 104 includes sensors for determining the ambient temperature, humidity, and light level. The environment monitor 104 will be discussed in greater detail below with reference to FIG. 4

The notification device 106, in one embodiment, includes a display 110 connected with a processing device. For example, the notification device 106 may be a portable electronic device such as a smartphone. In another embodiment, the notification device 106 is a media player that has the capability of communicating over a network 108. The notification device 106 is configured to receive personal patient indicators and environmental indicators that are useful in detecting the onset of an asthma attack. The notification device 106 may be configured with a patient manager 112.

The patient manager 112 is configured to receive input from the personal monitor 102 and the environment monitor 104 and determine a risk factor for the patient based on the input. The patient manager 112 may assign a score to the input based on patient benchmarks. For example, the patient manager 112 may assign a greater weighted score to certain indicators based on a patient history. Some of the factors that may be used in assigning the score include, but are not limited to:
age and height of the patient;
patient best rate of air flow;
types of medication,
   controller (i.e., maintenance), and
   rescue (i.e., last resort to stop asthma attack);
physical activity information (exercise);
what does the patient think triggers an attack;
whether the patient exposed to tobacco smoke, chemical fumes, dust, etc.;
whether the patient takes supplements/medicines;
occupation;
whether the patient has birds;
whether the patient has other animals;
whether the patient has recurrent wheezing;
whether the patient has coughing;
whether the patient has trouble breathing;
whether the symptoms worsen at night;
whether symptoms are triggered by cold air or exposures to allergens;
if the patient is a child:
   whether the child breathes louder than normal;
   breaths per minute (newborn 30-60/m) Toddlers are (20-40/m);
   whether the child has a frequent cough, clear mucus, or a runny nose caused by hay fever;
   whether the child frequently misses school; and
whether the child is limited in their participation in physical activities.

Thresholds may be assigned to different risk categories. The risk categories may be divided into low, medium, or high risk categories, with each category having a threshold value. For example, on a scale of 1-100, scores in the range of about 1-33 may be assigned to the low risk category, 34-66 to the medium risk category, and 67-100 to the high risk category. In an alternative embodiment, the categories may be in number greater or less than the above describe low/medium/high risk categories.

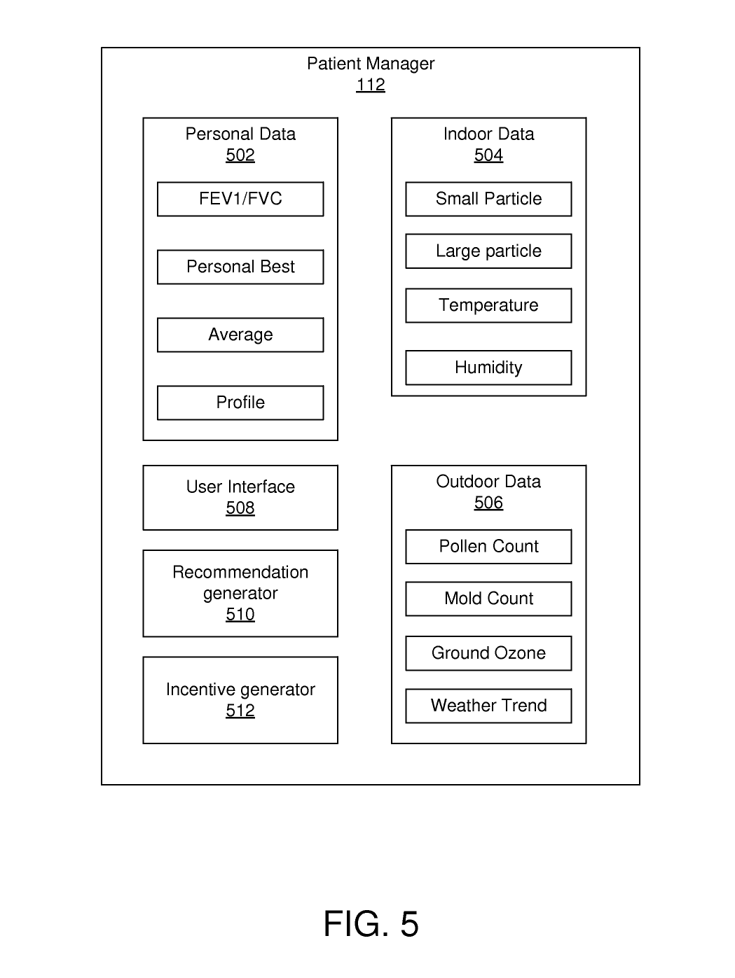

The patient manager 112 is configured to evaluate the risk factors in light of the current personal indicators from the personal monitor and the environment indicators from the environment monitor. The patient manager 112 stores a patient profile. The patient profile contains any information that is useful in determining the risk factor of the patient. For example, the above described risk factors may be stored in the patient profile. The patient manager 112 also is configured with a recommendation manager for generating an activity recommendation for the patient based on the identified risk category. The patient manager 112 will be discussed in greater detail below with reference to FIG. 5

In a further embodiment, the patient manager 112 may communicate over the network 108 with a patient server 114. The patient server 114 may be a datastore for maintaining profiles of many different patients. In another embodiment, the patient server 114 is configured to execute the patient manager 112. For example, the patient server 114 may receive input from the personal monitor 102 and the environment monitor 104 and generate an activity recommendation based on the input, which is then communicated to the notification device 106 and displayed on the display 110.

The patient manager 112, in one embodiment, is capable of communicating with an external data provider 116. The external data provider 116 supplies information related to allergens in the geographic region of the patient. The information may include, for example, pollen counts for trees, and mold, dust content in the air, etc., as well as temperature, humidity, and other weather forecast information. The patient manager 112 is configured to retrieve the information and, in one embodiment, utilize the information in generating activity recommendations. The patient manager 112 may perform simple GET requests to retrieve the information. In another embodiment, the external data may be formatted as an RSS feed, which the patient manager 112 retrieves based on a schedule.

FIG. 2 is a perspective view diagram illustrating one embodiment of the personal monitor 102, according to embodiments of the present disclosure. The personal monitor 102, in one embodiment, is formed having an inlet port 202 and an outlet port 204. The personal monitor 102 is configured to function as a spirometer. In other words, the personal monitor 102 is configured to measure lung function, particularly, the volume and/or speed of air that can be inhaled and exhaled. The personal monitor 102 may determine a forced volume vital capacity (“FVC”) and a forced expiratory volume (“FEV1”). FVC refers to a determination of the vital capacity from a maximally forced expiratory effort. FEV1 refers to a volume of air that has been exhaled at the end of a first second of forced expiration. The personal monitor 102 is configured to receive, at the inlet port 202, an exhaled volume of air, direct the air through an internal chamber of the personal monitor 102 and out the outlet port 204. A spirometer, or internal mass flow sensor, measures lung capacity.

Obtaining the FVC and the FEV1 from child patients is difficult. However, the patient manager 112 is configured to “gamify” the lung capacity test. Gamify, as used herein, refers to the use of game mechanics in a non-game context to engage the child patient and obtain useful spirometer readings. For example, the patient manager 112 may be configured to render a game where the child blows in to the personal monitor 102 to launch a projectile within a game operating on the notification device 106. This specific example is not intended to be limiting of the types of games or incentives that may be provided to the child to encourage lung capacity tests.

The personal monitor 102 may include a grouping of buttons 206. The buttons 206 may be configured to receive input from the patient and transmit the input to the patient manager 112. In this manner, the buttons may be used as input to a game, or as input to start and stop lung capacity tests. The buttons 206 may be programmable for receiving input, or alternatively, for displaying an indicator, such as a power indicator.

FIG. 3 is a perspective view diagram illustrating another embodiment of the personal monitor 102, in accordance with one embodiment of the present disclosure. The personal monitor 102, as described above, includes an apparatus for measuring lung capacity. In one embodiment, the apparatus is a spirometer for measuring the volume of air inspired and expired by the lungs using a precision differential pressure transducer. Types of spirometers include, but are not limited to, whole body plethysmographs, pneumotachometers, windmill-type spirometers, and tilt-compensated spirometers.

In another embodiment, the spirometer is a peak flow meter 302. The peak flow meter 302 is configured to measure the maximum speed of expiration of the patient, and accordingly, the degree of obstruction in the airways of the patient. The patient blows through the inlet port 202 of the personal monitor, and the expirated air passes through the peak flow meter 302 and out the outlet port 204. The peak flow meter 302 readings may vary across a wide range of values depending upon patient characteristics including sex, age, height, etc. In an alternative embodiment, the personal monitor 102 includes any type of sign (or symptom) measurement device desired to treat a specific disease. For example, the personal monitor may include a device for measuring glucose levels in blood.

The personal monitor 102 includes a storage device 304 for maintaining a history of peak flow meter values. The storage device 304, in one embodiment, is non-volatile storage device capable of storing peak flow meter 302 values.

The personal monitor 102, in one embodiment, includes a network interface controller 306 coupled with the storage device 304. The network interface controller 306, for example, is a wireless controller for connecting to a wireless network (e.g., Wi-Fi, or cellular). In an alternative embodiment, the network interface controller 306 is configured to establish a peer-to-peer connection with other devices described above with reference to FIG. 1. The network interface controller 306 is configured to communicate peak flow meter values with the patient manager 112.

FIG. 4 is a block diagram illustrating one embodiment of the environment monitor 104, in accordance with one embodiment of the present disclosure. The environment monitor 104 may be a box with an integrated plug for inserting into a power outlet on a wall. As such, the environment monitor 104 is an unobtrusive, all-in-one monitoring solution for determining ambient conditions in the room of the patient. Multiple environment monitors 104 may be deployed throughout a house, each of the environment monitors 104 configured to communicate with the patient manager 112.

The environment monitor 104, in one embodiment, includes a temperature sensor 402, a humidity sensor 404, an oxygen sensor 406, a network interface controller 408, a particle counter 410, a storage device 412, a battery backup 414, a sound detector 416, a photocell 418, and a night light 420. The environment monitor 104 may also include LEDs (not shown) that are indicative of the operating status of each of the above mentioned devices.

The temperature sensor 402, in one embodiment, is configured to determine the ambient temperature of the room or area in which the environment monitor 104 is placed. Examples of suitable temperature sensor 402 for use in the environment monitor 104 include, but are not limited to, a thermometer, thermistor, thermocouple, or resistance thermometer. Change in temperature can be a trigger for an asthma attack depending upon the patient, in particular, a drop in temperature can cause the onset of an asthma attack.

The humidity sensor 404 is configured to determine the humidity in the area surrounding the environment monitor 104. The humidity sensor 404 also determines the humidity trend, or stated differently, whether the humidity is increasing over time, decreasing over time, or remaining relatively flat. The humidity sensor 404, in one embodiment, is a hygrometer, examples of which include, but are not limited to, capacitive humidity sensor, resistive humidity sensor, and thermal conductivity humidity sensor. Like temperature, a change in moisture content in the air can be a trigger for an asthma attack.

The oxygen sensor 406 is configured to determine the proportion of oxygen in the air around the environment sensor. The oxygen sensor 406 may use technologies including, but not limited to, zirconia, electrochemical (i.e., galvanic), infrared, ultrasonic, or laser.

The network interface controller 408, for example, is a wireless controller for connecting to a wireless network (e.g., Wi-Fi, or cellular). In an alternative embodiment, the network interface controller 408 is configured to establish a peer-to-peer connection with other devices described above with reference to FIG. 1. The network interface controller 408 is configured to communicate any of the values determined by the sensors, devices, and counters of the environment monitor 104. The network interface controller 408 communicates the data with the patient manager 112, which may be executing on the notification device 106, or the patient server 114.

The particle counter 410 is configured to determine the size and quantity of particulate matter in the ambient air of the room. The particle counter 410, in one embodiment, is positioned in a channel formed in the interior of the environment monitor 104. A fan may be used to circulate air through the particle counter 410. The particle counter 410 may be based upon either light scattering, light obscuration, or direct imaging. As a particle passes through a light source of the particle counter 410, the redirected light is detected by a photo detector. The amplitude of light scattered or light blocked is measured and the particle is counted and tabulated. The environment monitor 104 is configured to identify when the air in the room is polluted to the point that the air may trigger an asthma attack. The environment monitor 104 may be programmed with a predetermined particle count threshold, or alternatively, the environment monitor 104 may receive from the patient manager 112 an indication of particle counts that have in the past led to the onset of an asthma attack.

The storage device 412, as with the storage device described above with reference to FIG. 3, is a non-volatile storage device for maintaining values obtained by the various sensors and counter of the environment monitor 104. The environment monitor 104 is configured to write to and read from the storage device 412 when communicating with the patient manager 112.

The battery backup 414 is configured to maintain the operating status of the environment monitor 104 even in the event of a power loss. The battery backup 414, in one embodiment, comprises an integrated battery within the environment monitor 104. In an alternative embodiment, the battery backup 414 is external to and attaches with the environment monitor 104.

The sound detector 416 is configured to identify and monitor sound levels in the room. The sound detector 416, in one embodiment, is a microphone or other sensor for the assessment of sound pressure levels. Changes in sound may lead to a change in stress levels in the patient, which in turn, may lead to the onset of an asthma attack. The photocell 418 is configured to identify light levels in the room and may be configured to energize the night light 420.

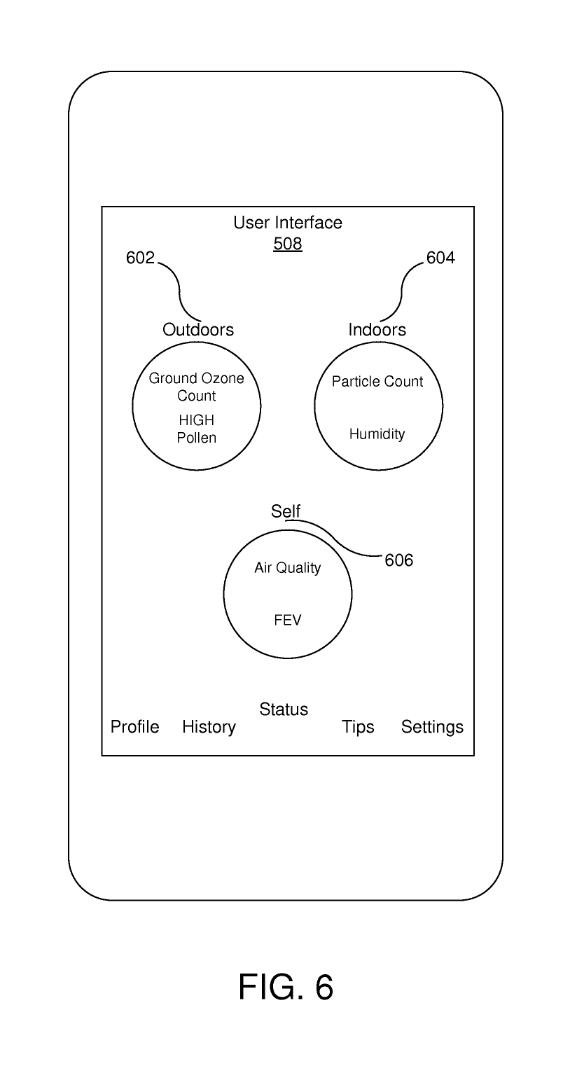

FIG. 5 is a schematic block diagram illustrating one embodiment of the patient manager 112, in accordance with embodiments of the present disclosure. The patient manager 112 is configured to generate incentives and activity recommendations based on personal data 502 from the personal monitor 102, indoor data 504 from the environment monitor 104, and outdoor data 506 from the external data provider 116. The incentives and activity recommendations are presented to the patient via a user interface 508 which may be displayed on the notification device 106. The patient manager 112 is operable on the notification device 106 as an application. Alternatively, the patient manager 112 may be configured to execute on the patient server 114, and accessed over the network 108. The user interface 508 will be described in greater detail below with reference to FIGS. 6-9.

The patient manager 112 is configured to maintain and analyze the personal data 502, the indoor data 504, and the outdoor data 506. Each of the three categories of data include triggers that may lead to an asthma attack. The patient manager 112 monitors each of the triggers and compares them to threshold values to determine when to make activity recommendations. The personal data 502 includes information gathered from the personal monitor 102 and the patient profile as described above with reference to FIG. 1. For example, the patient profile includes information regarding the age, height, weight, geolocation, etc., of the patient together with what factors tend to trigger an asthma attack. The patient manager 112 is configured to use the patient profile as a baseline for evaluating the triggers and generating recommendations. Over time, the patient manager 112 updates the profile based on recorded values of personal and environmental variables associated with what the patient determines to be a “good day,” and what the patient determines to be a “bad day.”

As described, the personal data 502 includes information collected as part of the profile together with information received from the personal monitor 102. The information includes, but is not limited to, personal best values (FEV1, FVC) for peak air flow as measured by the spirometer or peak flow meter. The patient manager 112 also maintains an average for the peak air flow.

The indoor data 504 is received from the environment monitor 104 and includes information related to the room in which the environment monitor 104. The patient manager 112 analyzes the environment information to determine trigger values such as, but not limited to, small particle count, large particle count, temperature, humidity, etc. The patient manager 112 also monitors the “trend” of the particle, temperature, and humidity information to determine if the ambient conditions of the room are trending towards or away from conditions that might lead to an asthma attack for the patient.

The outdoor data 506 is received from the external data provider 116, for example, and includes information including, but not limited to, pollen count, mold count, ground ozone levels, weather trends, dust levels, etc. The patient manager 112 is configured to analyze the outdoor data 506 to determine when to generate a recommendation to the patient to go exercise outdoors, and when to remain indoors. Each of the above described triggers may be assigned a weight that is used in determining an overall risk score. The weight may be determined according to the profile, or as a result of analyzing the data. For example, if the patient (or caregiver) has indicated in the profile that cold temperature is more likely to lead to an asthma attack than high pollen counts, the temperature values of the indoor and outdoor data may be given greater importance when determining the risk score.

The recommendation generator 510 is configured to generate an activity recommendation based on the risk score. Examples of activity recommendations may include, but are not limited to, “go play soccer,” or “play with a puzzler inside.” The recommendations may be customizable, or predetermined.

The incentive generator 512 is configured to generate incentives, or “gamify” the exercise of the patient's lungs. Over time, an asthma patient may increase their lung capacity through exercise, such as aerobic exercise. This exercise must be performed, however, on a “good day.” The patient manager 112 determines when the patient is having a good day based on a comparison of current data and average data. For example, the patient manager 112 compares the current peak flow values to the average peak flow value, current temperature to average temperature, etc., to determine if the conditions are ideal for the patient to exercise and increase their lung capacity. The incentive generator 512, in one embodiment, generates game-based scenarios to motivate the patient. For example, the incentive generator 512 may entice the patient to practice blowing into the personal monitor 102 by using the blowing action as part of a game, such as, launching projectiles at an enemy.

FIG. 6 is a schematic block diagram illustrating one embodiment of the user interface 508, in accordance with embodiments of the present disclosure. The user interface 508 may present in a useful manner information to the patient or a caregiver related to the management of asthma, or other diseases. The caregiver may be a parent, or a doctor. The user interface 508, while being depicted here on a smartphone, may be displayed in an application on a computing device, or through a web interface. More than one caregiver may monitor the asthma management of the patient. In one example, the user interface 508 displays the three main categories of asthma triggers, outdoors 602, indoors 604, and self 606 or personal.

The user interface 508 is configured to present the information maintained by the patient manager 112 as described above with reference to FIG. 5. The three data categories 502, 504, 506, are presented by the user interface 508 to the patient or caregiver. The user interface 508 may indicate to the caregiver whether any of the environmental or personal variables are within an acceptable range, or if they are above an acceptable threshold and immediate action should be taken. For example, if the outdoors 602 pollen count is above average, the user interface 508 may indicate to the patient or caregiver a “HIGH” notification. Furthermore, the user interface 508 may utilize colors to enhance the warning. For example, the “HIGH” notification may be emphasized with a red background.

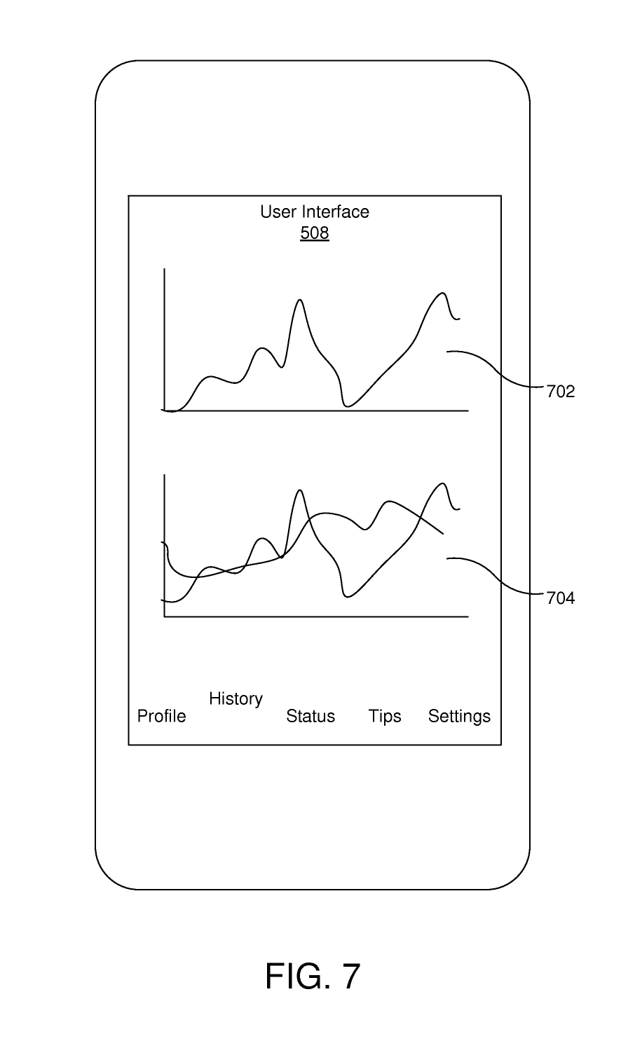

FIG. 7 is a schematic block diagram illustrating another embodiment of the user interface 508 in accordance with embodiments of the present disclosure. In one embodiment, the user interface 508 is configured to present a history of any selected personal or environmental variable as depicted in graph 702. For example, graph 702 may depict spirometer (or peak flow) readings over the past 30 days. The user interface 508 is also configured to compare two or more personal or environmental variables, as in graph 704 which is illustrative of spirometer values compared to particle counts.

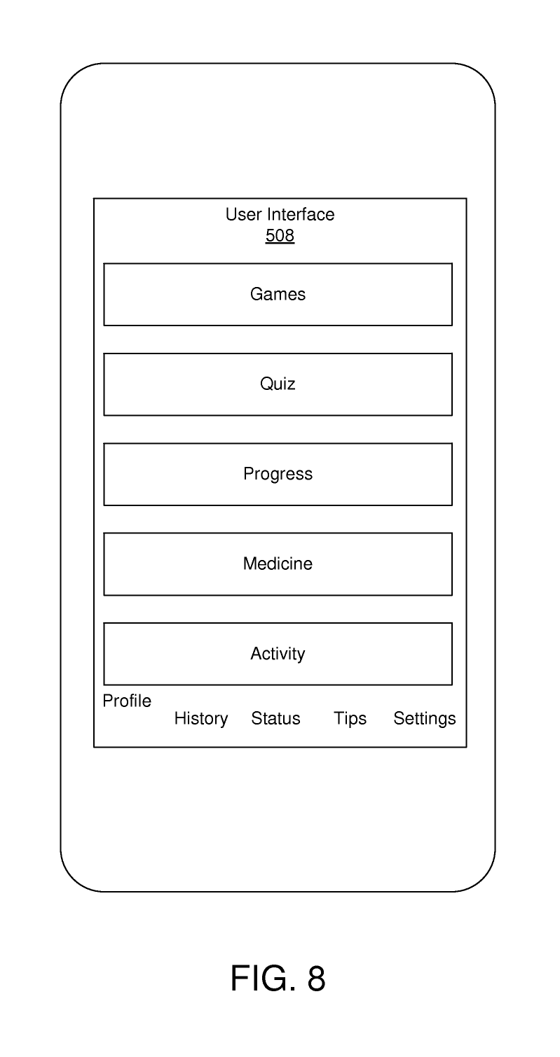

FIG. 8 is a schematic block diagram illustrating another embodiment of the user interface 508, in accordance with embodiments of the present disclosure. The user interface 508 may provide a mechanism for entering and updating the patient profile. The patient profile, in addition to the profile described above, may include preferences related to games, quizzes, progress, medicine, and activity. The patient manager 112 maintains information related to the patient profile, and in particular tracks the patient's medication schedule to ensure proper medication for the management of asthma.

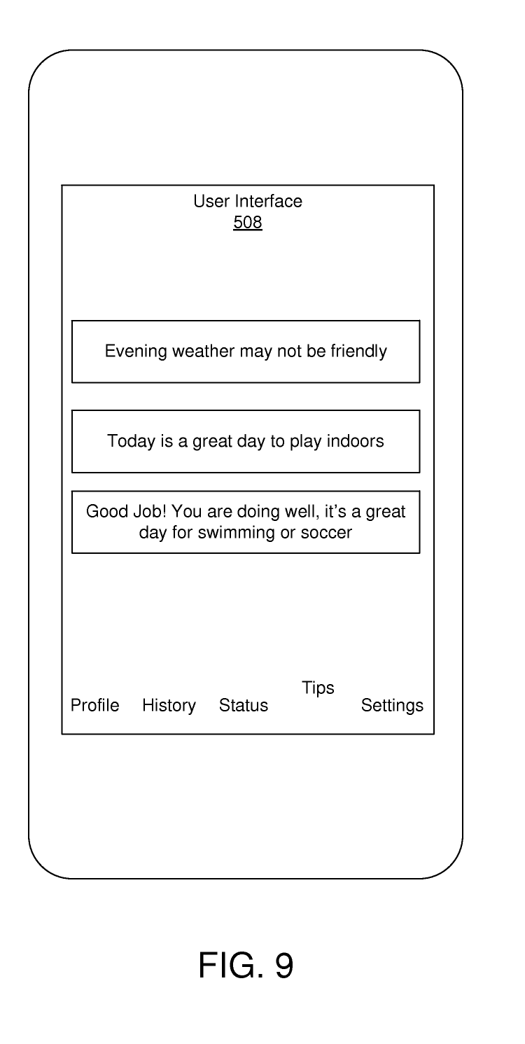

FIG. 9 is a schematic block diagram illustrating another embodiment of the user interface 508, in accordance with embodiments of the present disclosure. The user interface 508, in one embodiment, is configured to present activity recommendations according to the measured personal and environmental variables. As described above, the patient manager 112 analyzes the personal and environmental variables, and the recommendation generator 510 creates at least one recommendation based on a calculated risk score. The recommendations may include, but are not limited to, “evening weather may not be friendly,” “today is a great day to play indoors,” and “Good Job! You are doing well, it's a great day for swimming or soccer.”

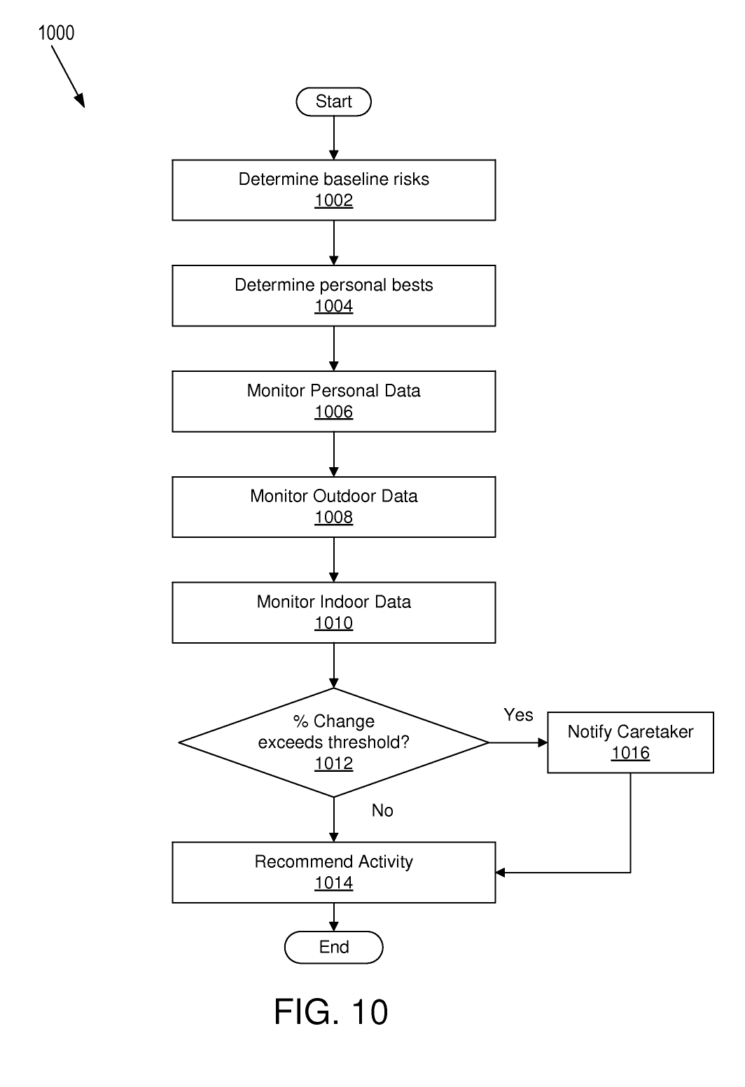

FIG. 10 illustrates a flow diagram of one embodiment of a method 1000 for generating activity recommendations. The method is performed by processing logic that may comprise hardware (circuitry, dedicated logic, etc.), software (such as is run on a general purpose computer system or a dedicated machine), or a combination of both. In one embodiment, the method is performed by a patient manager (e.g., patient manager 112 of FIG. 1).

The method 1000 starts and the processing logic, at block 1002, determines baseline risks. In one embodiment, the processing logic identifies the baseline risks based on the personal profile. The personal profile may have recommendations from the caregiver (parent or doctor) of the patient. The processing logic then, at block 1004, determines personal best values. For example, the processing logic is configured to determine a personal best peak flow or spirometer value for the patient.

At block 1006, the processing logic monitors the personal data received from the personal monitor 102. Examples of information received from the personal monitor 102 include, but are not limited to, lung capacity information measured by the spirometer.

At block 1008, the processing logic monitors outdoor data. In one embodiment, the processing logic receives outdoor data from an external data provider 116 as described above with reference to FIG. 1. The processing logic receives information including, but not limited to, temperature, pollen count, humidity, pollution levels, etc.

Likewise, at block 1010, the processing logic monitors indoor data. The indoor data is received from the environment monitor 104 and includes information related to the patient's room. For example, the processing logic monitors the temperature, humidity, and particulate count in the room.

The processing logic, at decision block 1012, determines if a percent change exceeds a threshold. The percent change refers to a combination of the daily or current value compared to the personal best value. The threshold, and the percent change depend on the patient and are customizable depending on whether the patient is a low, medium, or high risk patient. For example, a low risk patient may have an acceptable percent change in the range of about 20-30% while a high risk patient should be notified of a percent change of only 5%.

The processing logic may determine current or daily values by prompting the patient to blow into the personal monitor 102, or by “gamifying” the experience. For example, the processing logic may be coupled with a house controller and enable the patient to activate a light or appliance by blowing into the personal monitor 102. The processing logic may be configured to incorporate usage of the personal monitor into many daily activities, such as turning the page of an electronic book. Many different examples are contemplated, and one of skill in the art would readily identify numerous ways of incorporating usage of the personal monitor into daily life.

If the percent change does not exceed the threshold, the processing logic, at block 1014, recommends an activity. If the percent change does exceed the threshold, the processing logic at block 1016 notifies a caretaker before recommending an activity. The processing logic may recommend any activity that helps in managing lung capacity and asthma attacks according to the patient's asthma action plan. Examples include, but are not limited to, “take rescue inhaler, retest in 15 minutes,” “take a nap,” “go swimming,” “take corticosteroids twice a day, retest after one day,” etc. The method 1000 then ends.

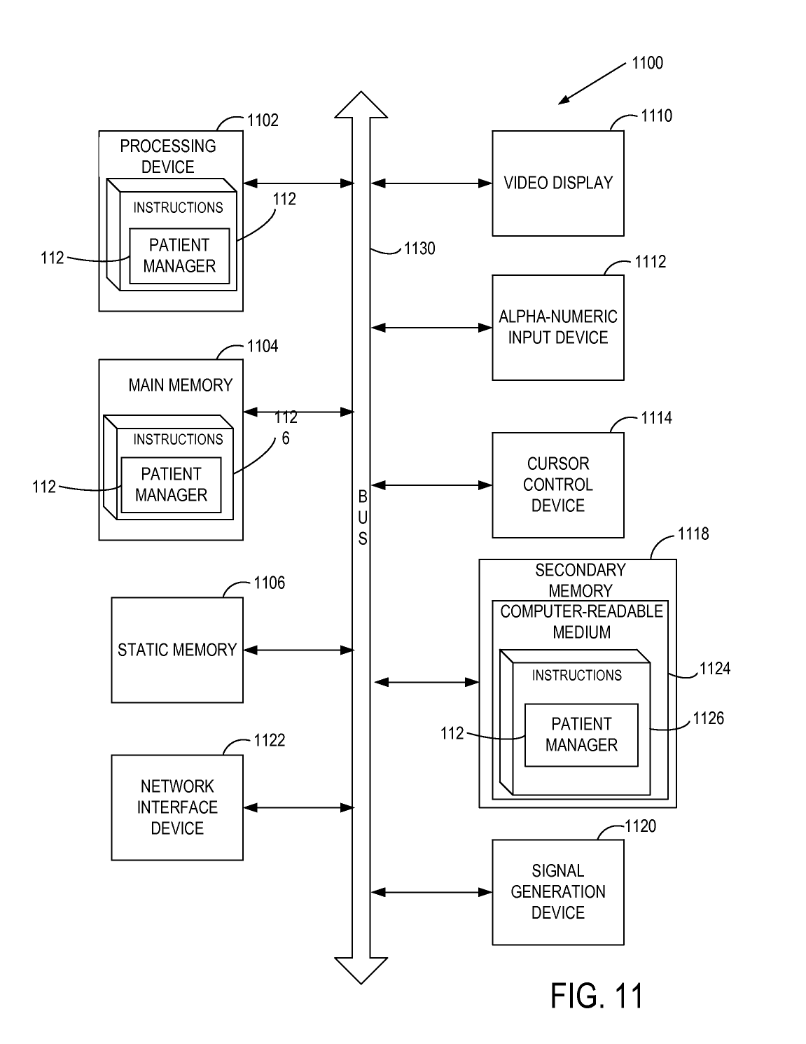

FIG. 11 is a diagram of one embodiment of a computer system for generating patient activity recommendations. Within the computer system 1100 is a set of instructions for causing the machine to perform any one or more of the methodologies discussed herein. In alternative embodiments, the machine may be connected (e.g., networked) to other machines in a LAN, an intranet, an extranet, or the Internet. The machine can be a host in a cloud, a cloud provider system, a cloud controller or any other machine. The machine can operate in the capacity of a server or a client machine in a client-server network environment, or as a peer machine in a peer-to-peer (or distributed) network environment. The machine may be a personal computer (PC), a tablet PC, a console device or set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein.

The exemplary computer system 1100 includes a processing device 1102, a main memory 1104 (e.g., read-only memory (ROM), flash memory, dynamic random access memory (DRAM) such as synchronous DRAM (SDRAM) or DRAM (RDRAM), etc.), a static memory 1106 (e.g., flash memory, static random access memory (SRAM), etc.), and a secondary memory 1118 (e.g., a data storage device in the form of a drive unit, which may include fixed or removable computer-readable storage medium), which communicate with each other via a bus 1130.

Processing device 1102 represents one or more general-purpose processing devices such as a microprocessor, central processing unit, or the like. More particularly, the processing device 1102 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 1102 may also be one or more special-purpose processing devices such as an application specific integrated circuit (ASIC), a field programmable gate array (FPGA), a digital signal processor (DSP), network processor, or the like. Processing device 1102 is configured to execute the instructions 1126 for performing the operations and steps discussed herein.

The computer system 1100 may further include a network interface device 1122. The computer system 1100 also may include a video display unit 1110 (e.g., a liquid crystal display (LCD) or a cathode ray tube (CRT)) connected to the computer system through a graphics port and graphics chipset, an alphanumeric input device 1112 (e.g., a keyboard), a cursor control device 1114 (e.g., a mouse), and a signal generation device 1120 (e.g., a speaker).

The secondary memory 1118 may include a machine-readable storage medium (or more specifically a computer-readable storage medium) 1124 on which is stored one or more sets of instructions 1126 embodying any one or more of the methodologies or functions described herein. In one embodiment, the instructions 1126 include instructions for the patient manager 112. The instructions 1126 may also reside, completely or at least partially, within the main memory 1104 and/or within the processing device 1102 during execution thereof by the computer system 1100, the main memory 1104 and the processing device 1102 also constituting machine-readable storage media.

The computer-readable storage medium 1124 may also be used to store the instructions 1126 persistently. While the computer-readable storage medium 1124 is shown in an exemplary embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing or encoding a set of instructions for execution by the machine and that cause the machine to perform any one or more of the methodologies of the present invention. The term “computer-readable storage medium” shall accordingly be taken to include, but not be limited to, solid-state memories, and optical and magnetic media.

The instructions 1126, components and other features described herein can be implemented as discrete hardware components or integrated in the functionality of hardware components such as ASICS, FPGAs, DSPs or similar devices. In addition, the instructions 1126 can be implemented as firmware or functional circuitry within hardware devices. Further, the instructions 1126 can be implemented in any combination hardware devices and software components.

In the above description, numerous details are set forth. It will be apparent, however, to one skilled in the art, that the present invention may be practiced without these specific details. In some instances, well-known structures and devices are shown in block diagram form, rather than in detail, in order to avoid obscuring the present invention.

Some portions of the detailed description which follows are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “providing,” “generating,” “detecting,” “analyzing,” “initializing,” “retrieving,” “identifying,” or the like, refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices.

In the preceding description, numerous details are set forth. It will be apparent, however, to one skilled in the art, that the present invention may be practiced without these specific details. In some instances, well-known structures and devices are shown in block diagram form, rather than in detail, in order to avoid obscuring the present invention.

Some portions of the detailed descriptions are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

The present invention also relates to an apparatus for performing the operations herein. This apparatus may be specially constructed for the required purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

The present invention may be provided as a computer program product, or software, that may include a machine-readable medium having stored thereon instructions, which may be used to program a computer system (or other electronic devices) to perform a process according to the present invention. A machine-readable medium includes any mechanism for storing or transmitting information in a form readable by a machine (e.g., a computer). For example, a machine-readable (e.g., computer-readable) medium includes a machine (e.g., a computer) readable storage medium such as a read only memory (“ROM”), random access memory (“RAM”), magnetic disk storage media, optical storage media, flash memory devices, etc.

Reference in the description to “one embodiment” or “an embodiment” means that a particular feature, structure, or characteristic described in connection with the embodiment is included in at least one embodiment of the invention. The phrase “in one embodiment” located in various places in this description does not necessarily refer to the same embodiment. Like reference numbers signify like elements throughout the description of the figures.

It is to be understood that the above description is intended to be illustrative, and not restrictive. Many other embodiments will be apparent to those of skill in the art upon reading and understanding the above description. Although the present invention has been described with reference to specific exemplary embodiments, it will be recognized that the invention is not limited to the embodiments described, but can be practiced with modification and alteration within the spirit and scope of the appended claims. Accordingly, the specification and drawings are to be regarded in an illustrative sense rather than a restrictive sense. The scope of the invention should, therefore, be determined with reference to the appended claims, along with the full scope of equivalents to which such claims are entitled.

We claim:

1. A method comprising:
receiving lung capacity information of a patient;
receiving environmental condition information for an inside area proximal to the patient; and
presenting, via a processor, an activity recommendation to the patient based on a comparison of the lung capacity information and the environmental condition information with baseline conditions.

2. The method of claim 1, further comprising receiving environmental condition information for an outside region proximal to the patient.

3. The method of claim 2, further comprising generating a risk score based on the lung capacity information, and the environmental condition information for the inside area and the outside area.

4. The method of claim 3, further comprising comparing the risk score with a baseline risk score and generating a percent change value, and further determining if the percent change value exceeds an acceptable threshold value.

5. The method of claim 4, wherein the acceptable threshold value is selected according to a patient profile.

6. The method of claim 4, further comprising notifying a caretaker when the percent change value exceeds the acceptable threshold value.

7. The method of claim 4, wherein the lung capacity information comprises a volume of air inspired and expired by lungs of the patient, and wherein the method further comprises updating a maximum volume of air value when the patient increases the volume of air.

8. The method of claim 7, wherein the percent change value is based on the baseline conditions and the maximum volume of air value.

9. A non-transitory computer readable storage medium including instructions that, when executed by a processing device, cause the processing device to perform a method comprising:
receiving lung capacity information of a patient;
receiving environmental condition information for an inside area proximal to the patient; and
presenting an activity recommendation to the patient based on a comparison of the lung capacity information and the environmental condition information with baseline conditions.

10. The computer readable storage medium of claim 9, wherein the method further comprises receiving environmental condition information for an outside region proximal to the patient.

11. The computer readable storage medium of claim 10, wherein the method further comprises generating a risk score based on the lung capacity information, and the environmental condition information for the inside area and the outside area.

12. The computer readable storage medium of claim 11, wherein the method further comprises comparing the risk score with a baseline risk score and generating a percent change value, and further determining if the percent change value exceeds an acceptable threshold value.

13. The computer readable storage medium of claim 12, wherein the acceptable threshold value is selected according to a patient profile.

14. The computer readable storage medium of claim 12, wherein the method further comprises notifying a caretaker when the percent change value exceeds the acceptable threshold value.

15. The computer readable storage medium of claim 12, wherein the lung capacity information comprises a volume of air inspired and expired by lungs of the patient, and wherein the method further comprises updating a maximum volume of air value when the patient increases the volume of air.

16. The computer readable storage medium of claim 15, wherein the percent change value is based on the baseline conditions and the maximum volume of air value.

17. A computing apparatus comprising:
a memory to store instructions for providing a patient manager; and
a computing device, coupled to the memory, wherein the computing device is configured to:
receive lung capacity information of a patient;
receive environmental condition information for an inside area proximal to the patient; and
present, via a processor, an activity recommendation to the patient based on a comparison of the lung capacity information and the environmental condition information with baseline conditions.

18. The computing apparatus of claim 17, wherein the computing device is further configured to receive environmental condition information for an outside region proximal to the patient.

19. The computing apparatus of claim 18, wherein the computing device is further configured to generate a risk score based on the lung capacity information, and the environmental condition information for the inside area and the outside area.

20. The computing apparatus of claim 19, wherein the computing device is further configured to compare the risk score with a baseline risk score and generating a percent change value, and further determining if the percent change value exceeds an acceptable threshold value.
